(19) United States
(12) Patent Application Publication
(10) Pub. No.: US 2004/0223939 A1
(43) Pub. Date: Nov. 11, 2004

(54) Cosmetic cleansing formulations based on a combination of sodium laureth sulfate and alkylpolyamphopolycarboxyglycinates

(75) Inventors: Andreas Clausen, Hamburg (DE); Ursula Jensen, Hamburg (DE); Stephan Ruppert, Hamburg (DE); Martin Sugar, Hamburg (DE); Maren Wilken, Norderstedt (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/776,665

(22) Filed: Feb. 10, 2004

Related U.S. Application Data
(63) Continuation of application No. PCT/EP02/08744, filed on Aug. 6, 2002.

(30) Foreign Application Priority Data
Aug. 10, 2001 (DE) 10139543.4
Oct. 11, 2001 (DE) 10150410.1

(51) Int. Cl. A61K 7/075; A61K 7/08

(52) U.S. Cl. 424/70.21; 424/70.24

(57) ABSTRACT

The present invention relates to the use of alkylpolyamphopolycarboxyglycinates for lowering the adsorption by the skin of sulfate and/or sulfonate surfactants during the cosmetic cleansing of the skin.

### CROSS REFERENCE TO RELATED APPLICATIONS

This is a continuation application of PCT/EP02/08744, filed Aug. 6, 2002, which is incorporated herein by reference in its entirety, and also claims the benefit of German Priority Application Nos. 101 39 543.4, filed Aug. 10, 2001 and 101 50 410.1, filed Oct. 11, 2001.

### FIELD OF THE INVENTION

The present invention relates to the use of a surfactant combination of sodium laureth sulfate and alkylpolyamphopolycarboxyglycinates in cosmetic cleansers.

### BACKGROUND OF THE INVENTION

Surfactants are of major importance as washing-active substances in cosmetic cleansers. As a result of their specific molecular structure having in each case one hydrophilic (water-attracting) and hydrophobic (water-repelling) group in the same molecule, they are able to reduce the surface tension of water, to wet the skin, to facilitate the removal of dirt and to regulate foam.

A distinction is made between four classes of surfactants:

A first class is the nonionic surfactants. These include fatty alcohol ethoxylates [RO(CH₂CH₂O)ₙH], fatty acid monoethanolamides [RCONHCH₂CH₂OH] and alkyl polyglycosides (APGs)

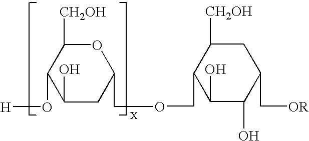

R=fatty acid radical.

A second class is the amphoteric surfactants. These are compounds which contain both a cationic function, in most cases a quaternary nitrogen, and an anionic function, in most cases a carboxylate group. They include alkylaminobetaines

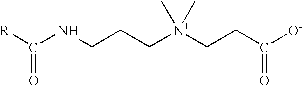

and also alkylamidoglycinates

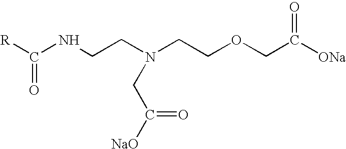

R=fatty acid radical

The group of cationic surfactants consists of compounds which contain at least one quaternary nitrogen atom. These include, for example, alkylamines, alkylimidazoles, ethoxylated amines.

The group of anionic surfactants is formed from sulfates, sulfonates and carboxylates, i.e. salts of esters of sulfuric acid, and also salts of sulfonic and carboxylic acids.

The most common surfactants from this class are derived from lauryl alcohol (C₁₂H₂₅OH). These are the sodium salt of lauryl sulfate (sodium lauryl sulfate, SLS) with the structure C₁₂H₂₅OSO₃ and ethylene glycol ether of the alcohol, which are likewise esterified with sulfuric acid (sodium laureth sulfate, SLES). These have the structure:

C₁₂H₂₅ (OCH₂CH₂)ₙ OSO3.

Standard commercial sodium lauryl ether sulfate (sodium polyoxyethylene lauryl sulfate) which has been used in the present invention is, however, usually a mixture of substances whose structures obey the general formula

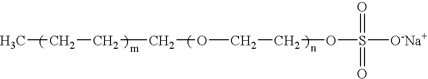

where m can assume the numbers 4 to 6 and n can assume the numbers 0 to 10.

These compounds have excellent washing-active properties and good foaming ability. However, in relatively high concentrations, they have an irritative effect on skin and mucosa. In addition, they lead to a reduction in skin moisture and to an increase in the transepidermal water loss (TEWL). Due to the good availability, the excellent washing properties and last but not least due to the acceptable cost, it is barely possible to dispense completely with this surfactant.

### SUMMARY OF THE INVENTION

It was therefore the object of the present invention to develop more skin-friendly cosmetic cleansing formulations based on sodium laureth sulfate.

Surprisingly, it was possible to overcome the shortcomings of the prior art using a mixture of the surfactants sodium laureth sulfate and one or more alkylpolyamphopolycarboxyglycinates, and to formulate cosmetic cleansers with good cleansing performance and high mildness. Although DE 19960767 also describes surfactant mixtures for reducing the adsorption by the skin of sodium laureth sulfate, this specification was unable to point the way to the present invention since it did not consider mixtures containing alkylpolyamphopolycarboxyglycinates whose effect in reducing the adsorption by the skin of sodium laureth sulfate is significantly greater than all hitherto known surfactant mixtures.

### BRIEF DESCRIPTION OF THE DRAWINGS

FIG. 1 is a graph illustrating the skin adsorption of the cosmetic cleansing preparations of the invention as compared to pure sodium laureth sulfate compositions.

FIG. 2 is a graph illustrating the skin adsorption of the cosmetic cleansing preparations of the invention as compared to formulations that include cocamidopropylbetaine.

### DETAILED DESCRIPTION OF THE PREFERRED EMBODIMENTS

The cosmetic cleansing formulations according to the invention comprise, in a preferred manner, sodium laureth sulfate in a concentration of from 1 to 20% by weight and particularly preferably in a concentration of from 5 to 15% by weight, based on the total weight of the formulation.

Moreover, the cosmetic cleansing formulations according to the invention comprise sodium carboxymethylcocoylpolypropylamine in a concentration of from 0.1 to 10% by weight, and particularly preferably in a concentration of from 1 to 5% by weight, based on the total weight of the formulation.

In addition, the cosmetic cleansing formulations can, in accordance with the invention, advantageously comprise:

anionic surfactants

acylamino acids (and salts thereof), such as

1. acyl glutamates, for example sodium acyl glutamate, di-TEA-palmitoyl aspartate and sodium caprylic/capric glutamate,

2. acylpeptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methylcocoyl taurate,

5. acyl lactylates, lauroyl lactylate, caproyl lactylate

6. alaninates,

carboxylic acids and derivatives, such as

1. carboxylic acids, for example lauric acid, aluminium stearate, magnesium alkanolate and zinc undecylenate,

2. ester carboxylic acids, for example calcium stearoyl lactylate, laureth-6 citrate and sodium PEG-4 lauramide carboxylate,

3. ether carboxylic acids, for example sodium laureth-13 carboxylate and sodium PEG-6 cocamide carboxylate,

phosphoric esters and salts, such as, for example, DEA-oleth-10 phosphate and dilaureth-4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂-₁₄-olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecyleneamido-MEA sulfosuccinate

and

sulfuric esters, such as

1. alkyl ether sulfate, for example sodium, ammonium, magnesium, MIPA, TIPA laureth sulfate, sodium myreth sulfate and sodium C₁₂-₁₃ pareth sulfate,

2. alkyl sulfates, for example sodium, ammonium and TEA lauryl sulfate.

B. Cationic Surfactants

Cationic surfactants which can optionally be used advantageously are

1. alkylamines,

2. alkylimidazoles,

3. ethoxylated amines and

4. quaternary surfactants,

5. ester quats.

Advantageous quaternary surfactants are alkylbetaine, alkylamidopropylbetaine and alkylamidopropylhydroxysulfane.

Cationic surfactants can also preferably be chosen for the purposes of the present invention from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxy-ethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds having cationic character, such as amine oxides, for example alkyldimethylamine oxides or alkylaminoethyldimethylamine oxides. In particular the use of cetyltrimethylammonium salts is advantageous.

C. Amphoteric Surfactants Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,

2. N-alkylamino acids, for example aminopropylalkylglutamide, alkylaminopropionic acid, sodium alkylimidodipropionate and lauroamphocarboxyglycinate.

D. Nonionic Surfactants Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated propoxylated lanolin, ethoxylated/propoxylated polysiloxanes, propoxylated POE ethers and alkyl polyglycosides, such as lauryl glucoside, decyl glycoside and cocoglycoside,

6. sucrose esters, sucrose ethers

7. polyglycerol esters, diglycerol esters, monoglycerol esters

8. methylglucose esters, esters of hydroxy acids.

Apart from the abovementioned substances, the compositions comprise, in accordance with the invention, optionally the additives customary in cosmetics, for example perfume, dyes, antimicrobial substances, refatting agents, complexing and sequestering agents, pearlescent agents, plant extracts, vitamins, active ingredients, preservatives, bactericides, pigments which have a coloring action, thickeners, softening, moisturizing and/or humectant substances, or other customary constituents of a cosmetic or dermatological formulation, such as alcohols, polyols, polymers, foam stabilizers, electrolytes, organic solvents or silicone derivatives.

In a particular embodiment, the present invention relates to liquid soaps or washing lotions. Such products are used not only for washing the hands, but are usually also used for the entire body, including the face. Accordingly, they are also suitable for use as shower preparation. In developing these products, the dermatological requirements are at the forefront since the skin is in intensive contact with the concentrated surfactant solution. Particular emphasis is therefore placed on the choice of mild surfactants in low concentration. Further criteria are also a good foaming ability, and a pleasant, refreshing scent and the simultaneous care of the skin. Washing lotions and in particular shower baths usually have viscosities of from about 3000 to 10000 mPa·s which, on the one hand, permit good extensibility of the product with rapid foaming, but, on the other hand, should be sufficiently high to enable trouble-free application by hand or flannels.

A further embodiment which is particularly preferred according to the invention relates to thickened preparations, as are used in shower gels and other high-viscosity cleansing formulations. According to the invention, the combination of ethoxylated glycerol isostearates and fatty alcohol polyglycol ethers is advantageous for thickening the surfactant systems according to the invention. Particular preference according to the invention is given here to the combination of PEG-90 glyceryl isostearate with laureth-2 (e.g. Oxetal VD92, Zschimmer & Schwarz). It is particularly advantageous according to the invention to use ethoxylated glyceryl isostearates in a concentration of from 0.2 to 8% by weight and in particular from 1 to 2% by weight, based on the total weight of the formulation, and to use fatty alcohol polyglycol ethers in a concentration of from 0.1 to 5% by weight and in particular from 0.1 to 0.5% by weight, based on the total weight of the formulation.

The ratio of alkyl ether sulfates +alkylpolyamphopolycarboxyglycinates to ethoxylated glycerol isostearates is, in these thickened preparations, 1:1 to 20:1 and particularly preferably 8:1 to 15:1. In these thickened preparations, the ratio of sulfate and/or sulfonate surfactants to alkylpolyamphopolycarboxyglycinates is 10:1 to 2:1 and particularly preferably 3:1 to 6:1.

Liquid soaps or washing lotions are generally characterized by a greater or lesser water content, but generally develop no noteworthy care effect since they have only a low oil content.

In addition, it is advantageous according to the invention to use the cosmetic cleansing formulations as bath, foam or shower preparation formulation and also as hair washing compositions (hair shampoo).

Furthermore, it is advantageous according to the invention to use the cosmetic cleansing formulations for reducing the adsorption by the skin of sodium laureth sulfate, for reducing the transepidermal water loss, and for increasing the skin moisture.

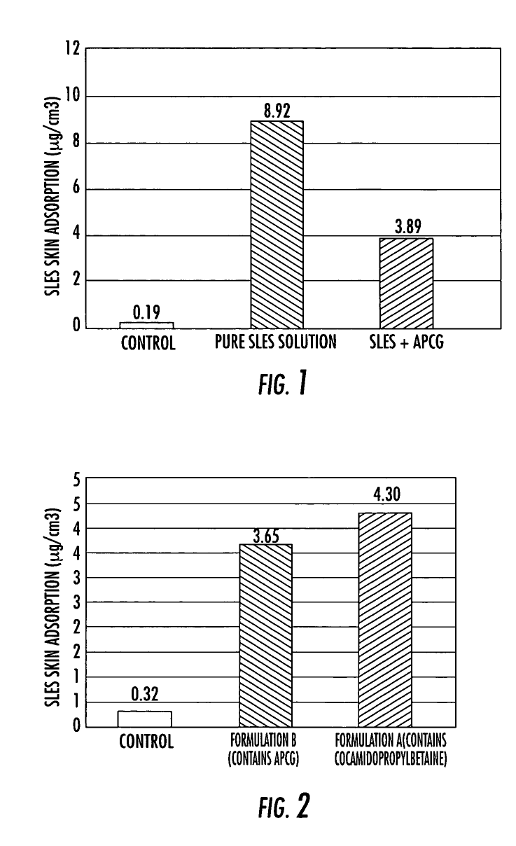

The superiority of the cosmetic cleansing preparations according to the invention with regard to the reduction in the adsorption by the skin of sodium laureth sulfate is shown by FIGS. 1 and 2. The examples below are intended to illustrate the present invention without limiting it. Unless stated otherwise, all amounts, proportions and percentages are based on the weight and the total weight of the preparations.

| | 1 | 2 | 3 | 4 | 5 |
|---|---|---|---|---|---|
| Sodium laureth sulfate | 10% | 12.8% | 14% | 9.1% | 9% |
| Sodium | 2.1% | 1.5% | 2% | 2.5% | 1.5% |
| carboxymethylcocoylpolypropylamine | | | | | |
| Cocoamidopropylbetaine | 1.65% | 3.3% | — | — | 2% |
| Sodium cocoyl glutamate | 1.25% | 0.75% | 1.75% | — | 1.5% |
| PEG-40 hydrogenated castor oil | 0.50% | 0.50% | 0.50% | 0.50% | 0.50% |
| PEG-100 hydrogenated glyceryl palmitate | 0.50% | 0.50% | 0.50% | 0.50% | 0.50% |
| Polyquaternium-10 | 0.05% | — | 0.2% | 0.1% | 0.15% |
| Sodium benzoate | 0.45% | 0.45% | 0.45% | 0.45% | 0.45% |
| Sodium salicylate | 0.20% | 0.20% | 0.20% | 0.20% | 0.20% |
| Citric acid | 0.50% | 0.50% | 0.50% | 0.50% | 0.50% |
| Perfume | q.s. | q.s. | q.s. | q.s. | q.s. |
| Water | ad | ad | ad | ad | ad |
| | 100 | 100 | 100 | 100 | 100 |
| | 6 | 7 | 8 | 9 | 10 |
| Sodium laureth sulfate | 10% | 12.8% | 14% | 9.1% | 10% |
| Sodium | 2.1% | 1.5% | 2% | 2.5% | 2% |
| carboxymethylcocoylpolypropylamine | | | | | |
| Cocoamidopropylbetaine | 1.65% | 3.3% | — | — | — |
| PEG-90 glyceryl isostearate | 1.0% | 0.5% | 0.5% | 1.7% | 1.1% |
| Laureth-2 | 0.11% | 0.055% | 0.055% | 0.2% | 0.12% |
| PEG-40 hydrogenated castor oil | 0.50% | 0.50% | 0.50% | 0.50% | 0.6% |
| PEG-100 hydrogenated glyceryl palmitate | 0.50% | 0.50% | 0.50% | 0.50% | — |
| Polyquaternium-10 | 0.05% | — | 0.2% | 0.1% | 0.2% |
| Sodium benzoate | 0.45% | 0.45% | 0.45% | 0.45% | 0.45% |
| Sodium salicylate | 0.20% | 0.20% | 0.40% | 0.20% | 0.4% |
| Citric acid | 0.50% | 0.50% | 0.50% | 0.50% | 0.9% |
| Benzophenone-4 | 0.05% | — | — | — | 0.05% |
| Styrene/acrylate/copolymer | — | 0.4% | — | 0.4% | — |
| EDTA | 0.2% | 0.2% | — | — | 0.2% |
| Perfume | q.s. | q.s. | q.s. | q.s. | q.s. |
| Water | ad | ad | ad | ad | ad |
| | 100 | 100 | 100 | 100 | 100 |

That which is claimed:

1. A cosmetic cleansing formulation, comprising:
a) one or more surfactants selected from the group consisting of sulfates and sulfonates, and
b) one or more alkylpolyamphopolycarboxyglycinates, said alkylpolyamphopolycarboxyglycinates present in an amount sufficient to reduce the adsorption by the skin of the one or more surfactants during cosmetic cleansing of the skin.

2. The cosmetic cleansing formulation as claimed in claim 1, wherein the one or more surfactants selected from the group consisting of sulfates and sulfonates include sodium laureth sulfate.

3. The cosmetic cleansing formulation as claimed in claim 2, wherein the one or more alkylpolyamphopolycarboxyglycinates include sodium carboxymethylcocoylpolypropylamine.

4. The cosmetic cleansing formulation as claimed in claim 2, wherein the sodium laureth sulfate is present in an amount between 1 and 20% by weight, based on the total weight of the formulation.

5. The cosmetic cleansing formulation as claimed in claim 2, wherein the sodium laureth sulfate is present in an amount between 5 and 15% by weight, based on the total weight of the formulation.

6. The cosmetic cleansing formulation as claimed in claim 2, wherein the one or more alkylpolyamphopolycarboxyglycinates are present in an amount between 0.1 and 10% by weight, based on the total weight of the formulation.

7. The cosmetic cleansing formulation as claimed in claim 2, wherein the one or more alkylpolyamphopolycarboxyglycinates are present in an amount between 1 and 5% by weight, based on the total weight of the formulation.

8. The cosmetic cleansing formulation as claimed in claim 2, further comprising one or more ethoxylated glycerol isostearates in a concentration of from 0.2 to 8% by weight, based on the total weight of the formulation.

9. The cosmetic cleansing formulation as claimed in claim 2, further comprising one or more fatty alcohol polyglycol ethers in a concentration of from 0.1 to 5% by weight, based on the total weight of the formulation.

10. The cosmetic cleansing formulation as claimed in claim 1, wherein the one or more alkylpolyamphopolycarboxyglycinates include sodium carboxymethylcocoylpolypropylamine.

11. The cosmetic cleansing formulation as claimed in claim 1, wherein the one or more surfactants are present in an amount between 1 and 20% by weight, based on the total weight of the formulation.

12. The cosmetic cleansing formulation as claimed in claim 1, wherein one or more surfactants are present in an amount between 5 and 15% by weight, based on the total weight of the formulation.

13. The cosmetic cleansing formulation as claimed in claim 1, wherein the one or more alkylpolyamphopolycarboxyglycinates are present in an amount between 0.1 and 10% by weight, based on the total weight of the formulation.

14. The cosmetic cleansing formulation as claimed in claim 1, wherein the one or more alkylpolyamphopolycarboxyglycinates are present in an amount between 1 and 5% by weight, based on the total weight of the formulation.

15. The cosmetic cleansing formulation as claimed in claim 1, further comprising one or more ethoxylated glycerol isostearates in a concentration of from 0.2 to 8% by weight, based on the total weight of the formulation.

16. The cosmetic cleansing formulation as claimed in claim 1, further comprising one or more fatty alcohol polyglycol ethers in a concentration of from 0.1 to 5% by weight, based on the total weight of the formulation.

17. A foam preparation including the cosmetic cleansing formulation as claimed in claim 1.

18. A hair shampoo including the cosmetic cleansing formulation as claimed in claim 1.

19. A shower gel including the cosmetic cleansing formulation as claimed in claim 1.

20. A formulation for application to the skin, comprising:
sodium laureth sulfate; and
sodium carboxymethylcocoylpolypropylamine.

21. The formulation as claimed in claim 20, wherein the sodium laureth sulfate is present in an amount from 9 to 15% by weight, based on the total weight of the formulation.

22. The formulation as claimed in claim 20, wherein the sodium carboxymethylcocoylpolypropylamine is present in an amount from 1 to 5% by weight, based on the total weight of the formulation.

23. A method of cleansing the skin, comprising applying to the skin a formulation comprising:
a) one or more surfactants selected from the group consisting of sulfates and sulfonates, and
b) one or more alkylpolyamphopolycarboxyglycinates.

24. The method as claimed in claim 23, wherein the one or more surfactants selected from the group consisting of sulfates and sulfonates include sodium laureth sulfate.
